(19) United States
(12) Patent Application Publication
(10) Pub. No.: US 2013/0309204 A1
(43) Pub. Date: Nov. 21, 2013

(54) VEGETABLE OILS AS NITROGEN FIXING BACTERIA PRESERVATIVE

(71) Applicants: Paul Edward Bonnett, Basel (CH); Carolyn Estep Moore, Greensboro, NC (US); Michael Burbach, Stanton, MN (US)

(72) Inventors: Paul Edward Bonnett, Basel (CH); Carolyn Estep Moore, Greensboro, NC (US); Michael Burbach, Stanton, MN (US)

(73) Assignee: SYNGENTA PARTICIPATIONS AG, Basel (CH)

(21) Appl. No.: 13/982,759

(22) PCT Filed: Feb. 2, 2012
(86) PCT No.: PCT/US12/23634

§ 371 Date: Jul. 31, 2013

(30) Foreign Application Priority Data
Feb. 2, 2011 (US) 61438843

(51) Int. Cl. A61K 35/74

(52) U.S. Cl. CPC A61K 35/74
USPC 424/93.4; 435/252.2

(57) ABSTRACT

Methods of preserving the life span of nitrogen-fixing bacteria in agricultural pesticide formulations using vegetable oils.

The present technology relates to chemical formulations for use in agricultural. More particularly the present technology relates to chemical formulations containing nitrogen fixing bacteria and at least one pesticide.

Nitrogen fixing bacteria are commonly used in the agricultural industries as an additive or treatment to promote nitrogen fixation within a given plant species. Microorganisms which carry out nitrogen fixation are known as diazotrophs. Diazotrophs are bacteria, or less commonly Archea, which fix atmospheric nitrogen into a usable form for other organisms, for example, plants. Diazotroph are generally broken into two groups. The first, free-living diazotrophs, includes, for example, anaerobes, facultative anaerobes, aerobes, oxygenic photosynthetic, and anoxygenic photosynthetic. The second, symbiotic diazotrophs, includes, for example, rhizobia, frankias, and cyanobacteria (e.g. blue-green algae or blue-green bacteria).

Plants that contribute to nitrogen fixation are generally legumes. A legume is a plant in the family Fabaceae or a fruit of those plants. Common legumes, include, but are not limited to, alfalfa, clover, peas, beans, lentils, lupins mesquite, carob, soy, and peanuts. Legumes have a symbiotic relations with rhizobia found in the root nodules of the plant. Growing legumes promotes nitrogen fixation in the soil and reduces input costs (e.g. fertilizer) to farmers, and therefore , legumes are commonly used in crop rotation to replenish soil of depleted nitrogen. In addition to legumes there are non-leguminous plants that can fix nitrogen, called actinorhizal plants.

As discussed above, nitrogen fixing bacteria are available as an additive or treatment to promote nitrogen fixation within a given plant species. The most commonly used bacteria used in the agricultural industry are rhizobia, which are used in soybeans and other legumes. Rhizobia based formulations are known and used in the industry. Two know formulations are Optimize™ available from EMD Crop BioSciences of Brookfield, Wis. and Vault® available from Becker Underwood of Ames, Iowa.

Rhizobia based formulations such as Optimize™ and Vault® contain the rhizobia bacterial strain Bradyrhizobium japonicum, a strain known for its ability to facilitate nitrogen fixation in legumes. These formulations contain a given concentration of rhizobia and are formulated to provide food for the bacteria (e.g. sugar) and to keep the bacteria alive. However, rhizobia based formulations are commonly combined with an agricultural pesticide formulation, for example seed treatments, prior to being applied to the plant or the plant seed. Agricultural pesticide formulations can have an adverse effect on the rhizobia based formulations once combined, and can result in the accelerated death of the rhizobia; lowering the rhizobia concentration of the mixture.

The present technology provides for a formulation and method of preserving and/or extending the life span of rhizobia in an agricultural pesticide formulation. The present technology provides for the the addition of a vegetable oil to an agricultural formulation comprising rhizobia and at least one pesticide to preserve and/or extend the life span of rhizobia in an agricultural pesticide formulation.

Vegetable oils are lipid materials derived from plants. Common examples of vegetable oils include oils such as canola oil, linseed oil, soybean oil, corn oil, safflower oil, palm oil, sunflower oil, peanut oil, cottonseed oil, palm kernel oil, rapeseed oil, and olive oil. One preferred embodiment is soybean oil.

Preferred target application rates to seeds for the addition of vegetable oils are 0.1-5.0 fl oz/cwt; 0.25-3.0 fl oz/cwt, 0.25-2.5 fl oz/cwt, 0.25-2.0 fl oz/cwt, 0.25-1.5 fl oz/cwt, 0.5-2.0 fl oz/cwt, and 0.5-1.0 fl oz/cwt.

Preferred mixing ratios of the agricultural formulations (AF) and the oils (O) are, in AF:O, from 10:1 to 1:10, 7.5:1 to 1:7.5, 5:1 to 1:5, 2:1 to 1:2, and 2:1 to 1:1. In certain embodiments, ratios are about 1:1, about 1.25:1, about 1.5:1, about 1.75:1, about 2:1, about 3:1, about 4:1, about 5:1, about 6:1, about 7:1, about 8:1, about 9:1, and about 10:1.

### EXAMPLES

CruiserMaxx® Beans (CMB) is a seed treatment containing the active ingredients thiamethoxam (25.55% w/v), mefenoxam (1.92% w/v), and fludioxonil (1.27% w/v) from Syngenta Crop Protection and was used as the base pesticide formulation. Optimize™ and Vault® were used as the rhizobia based formulations. Normal cooking oil labeled 100% pure soybean oil distributed by LaPreferida of Chicago, Ill. was used for the formulations which included soybean oil.

TABLE 1
Table 1: Treatment List

| Treatment | Components |
|---|---|
| 1 | CMB + Optimize ™ |
| 2 | CMB + Optimize ™ + Soybean Oil @ 0.5 fl oz/cwt seed |
| 3 | CMB + Optimize ™ + Soybean Oil @ 1.0 fl oz/cwt seed |
| 4 | CMB + Vault ® |
| 5 | CMB + Vault ® + Soybean Oil @ 0.5 fl oz/cwt seed |
| 6 | CMB + Vault ® + Soybean Oil @ 1.0 fl oz/cwt seed |
| 7 | Optimize ™ |
| 8 | Vault ® |

CMB application rate was 3.0 fl oz/cwt seed,
Optimize ™ application rate was 2.8 fl oz/cwt seed, and
Vault ® application rate was 2.0 fl oz/cwt seed.

Procedures

Each pesticide and/or pesticide/oil combination was combined with each of the two Inoculants at the recommended rates and applied to soybean seed immediately after mixing, and the rhizobia concentration per seed determined at 1 day, 7 days, 14 days, and 30 days.

The appropriate mixtures were prepared in test tube quantities and the proper amount of this mix was applied to one half pound of soybean seed in a Ziploc® bag. The seed and fungicide mix were thoroughly mixed in the bag and the seed then stored at room temperature (approximately 18-19° C.).

One milliliter aliquots of each mix were taken at the appropriate times, diluted in sterile phosphate buffered de-ionized water blanks, and plated at appropriate levels.

Ten seeds from each treated seed batch are taken at the appropriate times and likewise diluted in sterile phosphate buffered de-ionized water blanks—after being shaken or ‘extracted’ in a 100 ml buffered water blank for approximately 10 minutes.

Yeast mannitol agar medium was used for all platings. Plates are incubated for 7-10 days at 28° C.

TABLE 2
Table 2: Results of Survival on Seed

Rhizobia per Seed

| Treatment | 1 Day | 7 Days | 14 Days | 30 Days |
|---|---|---|---|---|
| 1 | 240,000 | 91,000 | 70,000 | 14,000 |
| 2 | 410,000 | 140,000 | 100,000 | 21,000 |
| 3 | 320,000 | 120,000 | 86,000 | 20,000 |
| 4 | 250,000 | 200,000 | 84,000 | 36,000 |
| 5 | 400,000 | 160,000 | 110,000 | 62,000 |
| 6 | 300,000 | 180,000 | 116,000 | 66,000 |
| 7 | 450,000 | 300,000 | 104,000 | 42,000 |
| 8 | 540,000 | 290,000 | 215,000 | 66,000 |

The term “pesticide” as utilized herein is intended to cover compounds active against pests which are intended to repel, kill, or control any species designated a pest including weeds, insects, rodents, fungi, bacteria, or other organisms.

Examples of individual compounds of the above mentioned compound classes are listed below. Where known, the common name is used to designate the individual compounds (q.v. the Pesticide Manual, 12th edition, 2001, British Crop Protection Council).

Examples of pesticides include those selected from, for example and not for limitation, insecticides, acaricides, bactericides, fungicides, nematicides and molluscicides.

Suitable additions of insecticidally, acaricidally, nematicidally, or molluscicidally active ingredients are, for example and not for limitation, representatives of the following classes of active ingredients: organophosphorus compounds, nitrophenols and derivatives, formamidines, triazine derivatives, nitroenamine derivatives, nitro- and cyanoguanidine derivatives, ureas, benzoylureas, carbamates, pyrethroids, chlorinated hydrocarbons and Bacillus thuringiensis products. Especially preferred components in mixtures are abamectin, cyanoimine, acetamiprid, thiodicarb, nitromethylene, nitenpyram, clothianidin, dinotefuran, fipronil, lufenuron, pyripfoxyfen, thiacloprid, fluxofenime; imidacloprid, thiamethoxam, Chloranthraniliprole, beta cyfluthrin, lambda cyhalothrin, fenoxycarb, diafenthiuron, pymetrozine, diazinon, disulphoton; profenofos, furathiocarb, cyromazin, cypermethrin, taufluvalinate, spinetoram, spinosad, sulfloxaflor, tefluthrin or Bacillus thuringiensis products, very especially abamectin, thiodicarb, cyanoimine, acetamiprid, nitromethylene, nitenpyram, clothianidin, dinotefuran, fipronil, thiacloprid, imidacloprid, thiamethoxam, chloranthraniliprole, beta cyfluthrin, lambda cyhalothrin, and tefluthrin.

Suitable additions of fungicidally active ingredients are, for example and not for limitation, representatives of the following classes of active ingredients: strobilurins, triazoles, ortho-cyclopropyl-carboxanilide derivatives, phenylpyrroles, and systemic fungicides. Examples of suitable additions of fungicidally active ingredients include, but are not limited to, the following compounds: azoxystrobin; bitertanol; carboxin; Cu₂O; cymoxanil; cyproconazole; cyprodinil; dichlofluamid; difenoconazole; diniconazole; epoxiconazole; fenpiclonil; fludioxonil; fluoxastrobin, fluquiconazole; flusilazole; flutriafol; furalaxyl; guazatin; hexaconazole; hymexazol; imazalil; imibenconazole; ipconazole; kresoxim-methyl; mancozeb; metalaxyl; mefenoxam; metconazole; myclobutanil, oxadixyl, pefurazoate; penconazole; pencycuron; penflufen, prochloraz; propiconazole; pyroquilone; (±)-cis-1-(4-chlorophenyl)-2-(1H-1,2,4-triazol-1-yl)cycloheptanol; sedaxane; spiroxamin; tebuconazole; thiabendazole; tolifluamide; triazoxide; triadimefon; triadimenol; trifloxystrobin, triflumizole; triticonazole and uniconazole. Particularly preferred fungicidally active agents include azoxystrobin, difenoconazole, penflufen, fludioxonil, thiabendazole, tebuconazole, metalaxyl, mefenoxam, myclobutanil, fluoxastrobin, tritaxonazole, sedaxane, and trifloxystrobin.

The form of the active ingredients may be selected so as to optimize the application or the bioperformance of the compositions. The forms of the active ingredient compositions may be selected, depending on these intended aims and the prevailing circumstances, from solutions, emulsifiable concentrates, suspension concentrates, directly sprayable or dilutable solutions, emulsions, microemulsions, suspo-emulsions, aqueous capsule suspensions, spreadable pastes, dilute emulsions, sprayable powders, soluble powders, dispersible powders, wettable powders, slurries, dusts, granules or encapsulations.

We claim:

1. A method of preserving the life span of nitrogen fixing bacteria in an agricultural pesticide formulation, the method comprising:
combining a nitrogen fixing bacteria preserving effective amount of a vegetable oil with an agricultural pesticide formulation, wherein said pesticide formulation comprises a pesticide and nitrogen fixing bacteria.

2. The method of claim 1, wherein said nitrogen fixing bacteria is rhizobia.

3. The method of claim 1, wherein said vegetable oil is soybean oil.

4. The method of claim 1, wherein said pesticide is thiamethoxam.

5. The method of claim 1, wherein said nitrogen fixing bacteria is rhizobia, wherein said vegetable oil is soybean oil, and wherein said pesticide is thiamethoxam.

6. The method of claim 1, wherein said nitrogen fixing bacteria are present in a concentration greater than 2×10⁷ nitrogen fixing bacteria per milliliter.

7. A method of bacterial safening in a pesticide formulation comprising:
combining a nitrogen fixing bacteria preserving effect amount of a vegetable oil with an agricultural pesticide formulation, wherein said pesticide formulation comprises a pesticide and nitrogen fixing bacteria.

8. A method of preserving the life span of nitrogen fixing bacteria on a plant seed, the method comprising:
applying, to a plant seed, a combination of a nitrogen fixing bacteria preserving effective amount of a vegetable oil with an agricultural pesticide formulation, wherein said pesticide formulation comprises a pesticide and nitrogen fixing bacteria.

9. The method of claim 8, wherein said nitrogen fixing bacteria is rhizobia.

10. The method of claim 8, wherein said vegetable oil is soybean oil.

11. The method of claim 8, wherein said pesticide is thiamethoxam.

12. The method of claim 8, wherein said nitrogen fixing bacteria is rhizobia, wherein said vegetable oil is soybean oil, and wherein said pesticide is thiamethoxam.

13. The method of claim 8, wherein the application rate of the oil to the plant seeds is from 0.25-3.0 fl oz/cwt.
